(19) United States
(12) Patent Application Publication
(10) Pub. No.: US 2011/0052921 A1
(43) Pub. Date: Mar. 3, 2011

(54) REACTIVITY OF FLY ASH IN STRONGLY AKLALINE SOLUTION

(75) Inventors: Weiliang GONG, Rockville, MD (US); Werner LUTZE, Chevy Chase, MD (US); Chen CHEN, Washington, DC (US); Ian PEGG, Alexandria, VA (US)

(73) Assignee: The Catholic University of America

(21) Appl. No.: 12/851,040

(22) Filed: Aug. 5, 2010

Related U.S. Application Data
(60) Provisional application No. 61/214,000, filed on Aug. 6, 2009.

(51) Int. Cl. B32B 9/00; G01N 33/00

(52) U.S. Cl. 428/426; 436/145; 428/689; 428/446; 428/454

(57) ABSTRACT

Provided in one embodiment is a method of charactering a fly ash composition, comprising determining a reactivity of the fly ash composition in a solution. The applicability of the findings to low water-to-solid ratios for the process of geopolymerization for the relationship between the amounts of fly ash reacted, and the compressive strength of a geopolymer cement is also described.

### CROSS-REFERENCE TO RELATED PATENT APPLICATIONS

This application claims priority from U.S. Provisional Application Ser. No. 61/214,000, filed Aug. 6, 2009, which is hereby incorporated herein by reference in its entirety.

All publications, patents, and patent applications cited in this Specification are hereby incorporated by reference in their entirety.

The articles “a” and “an” are used herein to refer to one or to more than one (i.e. to at least one) of the grammatical object of the article. By way of example, “a polymer resin” means one polymer resin or more than one polymer resin. Any ranges cited herein are inclusive. The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

### BACKGROUND

Fly ash is a byproduct of coal combustion. The annual world production exceeds 850 million tons, most of which is placed in landfills at significant cost. During storage, hazardous constituents may be leached out, reach the groundwater, and pose a risk to the environment and to humans. Use, rather than disposal, of fly ash should be the strategy for the future. Large amounts of fly ash can be used as a constituent of cement and concrete for the growing construction industry, e.g., in China and India, the world's largest producers of fly ash. In addition, unlike Portland cement, production of cement based on fly ash can be accomplished without generating carbon dioxide. Hence, the use of fly ash would have at least two environmentally beneficial effects. However, production and use of alternative or ‘green’ cement requires an understanding of the processes determining the short- and long-term materials properties of the final product, which are well known for conventional products such as Portland cement.

In previous work, fly ash and other pozzolanic material has been used in new waste forms for treatment of low-level radioactive waste and hazardous waste [1, 2], best described as geopolymers. After Davidovits pioneered research in the alkali activation of metakaolin [4-6], geopolymers have attracted increasing attention [7-12]. Review articles have been published recently by Komnitsas and Zaharaki [13], Duxson et al. [14], Khale and Chaudhary [15], and Pacheco-Torgal et al. [3, 16].

Geopolymer cements ('Geocement') based on fly ash are currently being developed for potential construction applications, wherein the underlying process is geopolymerization [17]. Frequently, geopolymers are made by mixing a highly alkaline silicate solution with an aluminum- and silicon-bearing oxide or other compound, typically metakaolin. Metakaolin is expensive and can be replaced by other pozzolanic materials or combinations of materials as long as they participate in the following reactions: 1) complete or partial dissolution or alteration of the aluminum- and silicon-bearing phase(s) in alkali silicate solution at high pH (pH>13); 2) quick release of aluminate and silicate species from the solid to the liquid phase between the reacting particles; 3) polymerization of silicate and aluminate species together with silicate already in solution and formation of a gel phase with chemical bonds to unreacted material; and 4) hardening of the gel phase by expulsion of water [14, 18-22]. The details of the mechanisms, which explain setting and hardening of a geopolymer, are still under investigation [3, 16].

Geopolymerization is a complex process involving a sequence of consecutive and parallel reactions comprising dissolution, diffusion, precipitation, and solidification reactions [23]. These reactions are analogous to those observed in zeolite synthesis from solid precursors, although in geopolymers reactions occur at much higher water-to-solid ratios [8]. The geopolymer gel structure is related to that of aluminosilicate gels from which zeolites form under hydrothermal conditions [9, 24, 25]. It is likely that a significant part of geopolymeric gels is composed of nanometer-size crystalline structures [26]. In some cases, zeolitic material has been detected in geopolymers [27-29]. Therefore, current models describing geopolymerization [22, 23] use information about zeolite synthesis in alkaline solutions, as well as from aluminosilicate dissolution and weathering. Unfortunately, because the water-to-solid mass ratio is small, e.g., <0.5 g/g, reactions before and during geopolymerization take place almost simultaneously, this makes studying these reactions individually very challenging. One way to ease the task is to use higher water-to-solid ratios, as is frequently done when studying hydrothermal synthesis of zeolites from fly ash in alkaline solution [30].

To develop new geopolymeric formulations with fly ash as a major component it is important to understand the relationship between the reactivity of fly ash and the materials properties of the final product (e.g., compressive strength). Efforts have been made to explore the processes involved in geopolymer product formation, [3, 13-16]; yet only a few authors have studied the processes explaining fly ash reactivity in geopolymer systems [27-29, 31-34]. In contrast, there is extensive literature in fly ash reactivity and reaction kinetics in fly ash/Portland cement systems. As an example, in fly ash/Portland cement systems fly ash reactivity is measured by the rate of reaction of fly ash with calcium hydroxide. Bumrongjaroen et al. [35] published a summary of methods measuring reactivity of fly ash in fly ash/Portland cement systems. Many researchers used conductometric techniques to monitor indirectly the depletion of lime in solution as an indicator of reaction progress. The assumption is that Ca²⁺ dominates conductivity [36]. Other methods detect the amount of lime reacted by measuring the release of heat and changes of mass. For example, the lime concentration can be determined chemically by solvent extraction and by thermogravimetry and differential thermal analysis [37-40].

In a number of studies of geopolymer systems, reactivity of fly ash and other pozzolanic materials has been evaluated by measuring how much SiO₂, Al₂O₃, CaO, and other constituents have dissolved in alkaline solution at various water-to-solid ratios, i.e., 1 to 2000 g/g [12, 31, 41-46]. Pietersen et al. [41] and Lee and van Deventer [43] conducted studies on the time dependence of dissolution of fly ash at different pH and temperatures. Published data quantify the mass of fly ash dissolved, while little was reported about precipitation of gel, zeolite crystallization, and conversion of the glass phase in fly ash into gel. No kinetic analysis has been reported.

### SUMMARY

One objective of the present invention is to provide a procedure to quantify reactivity of fly ashes in alkaline solutions as a function of pH and temperature. This procedure allows the study of dissolution and other processes individually at a higher water-to-solid ratio than generally used in geopolymer pastes. Described herein is the significance of the results for low water-to-solid ratios (e.g., 0.35, i.e., for the process of geopolymerization), and for the relationship between the relative mass of fly ash reacted and the compressive strength of a geopolymer cement. In one embodiment, the reactivity of the fly ash as determined by the method described herein can be further used to determine a mechanical property of at least one of (i) a geopolymer cement, (ii) Portland cement, and (iii) concrete matrix formed using the fly ash. The mechanical property can refer to any property, depending on the context. For example, it can refer to compressive property such as compressive strength.

One embodiment provides a method of characterizing a fly ash composition, comprising determining a reactivity of the fly ash composition in a solution. In the method, the determination can be carried out via at least one of (i) solution analysis and (ii) mass loss measurement. In one embodiment, the reactivity of the fly ash composition can be expressed in terms of (i) a relative mass of a glass phase of the fly ash dissolved, (ii) a relative mass of a glass phase of the fly ash converted into a gel, (iii) a relative mass of a material precipitated from the fly ash as at least one crystal, or combinations thereof.

Another embodiment provides a composition, comprising: (i) a core comprising a fly ash composition, (ii) a gel layer over at least a portion of the core, wherein the layer comprises at least one oxide, and (iii) a crust layer over at least a portion of the core, wherein the crust layer comprises at least one crystal.

In another embodiment, fly ash samples from six power stations were leached in potassium hydroxide solutions as a function of pH and temperature at a water-to-solid ratio of 40 g/g. Pristine fly ash was analyzed for composition, crystalline phases, and content of glass. From the results, a method was developed which provides for a detailed characterization of the reactivity of fly ash. The leaching process could be divided into three stages. In stage one, reaction progress measured by the relative mass of fly ash reacted (α), was controlled by the rate of glass dissolution, while very little gel formed (α<0.1). In stage two, more gel (oxides of Fe, Ca, Mg and Ti) formed on the glass surface and the rate of glass dissolution was limited by diffusion (0.1<α<0.45). In stage three, zeolite (Linde F) crystallized on top of the gel layer, and an aluminosilicate gel formed in situ, growing inward, while diffusion continued to control reaction progress (α>0.45). The data were modeled using a modified version of the Jander equation and rate constants were calculated. Activation energies are in agreement with typical values for other silicate glasses. The rate constants for stage one reflect an intrinsic glass property and chemical durability, which increased linearly with increasing concentration of network formers in the glass. The significance of the findings for low water-to-solid ratios (e.g., 0.35), for the process of geopolymerization, for the relationship between the amounts of fly ash reacted, and the compressive strength of a geopolymer cement was also described.

### BRIEF DESCRIPTION OF THE DRAWINGS

FIG. 1 shows the relative mass loss of Headwater fly ash in 7.5 M KOH at 40° C.

FIG. 2 shows the reaction progress of Headwater fly ash as a function of temperature in 7.5 M KOH.

FIG. 3 shows the reaction progress of Headwater fly ashes as a function of KOH molarity at 75° C.

FIG. 4 shows the reactivity of six different fly ashes in 7.5 M KOH at 75° C.

FIG. 5 shows the reactivity of six different fly ashes in 7.5 M KOH at 50° C.

FIG. 6 shows the XRD patterns of Headwater fly ash as a function of reaction progress; shown in the figure is evidence of crystallization of Linde F zeolite in 7.5 M KOH at 50° C.

FIG. 7 shows the SEI micrograph of Headwater fly ash particles after leaching in 7.5 M KOH solution at 75° C., α=0.62; inlay shows Linde F zeolite crystals.

FIG. 8 shows the SEI micrograph (cross-section) of Headwater fly ash particles after leaching in 7.5 M KOH at 75° C., α=0.62.

FIG. 9 shows the BEI micrograph (cross-section) of Headwater fly ash particles after leaching in 7.5 M KOH, α=0.71.

FIG. 10 shows the relative masses of major oxides in the leachate and in the gel as function of reaction progress (α) at 40° C., 7.5 M KOH.

FIG. 11 shows the relative mass of major oxides in the leachate and in the gel as a function of reaction progress (α) at 75° C., 7.5 M KOH.

FIG. 12 illustrates the analysis of reaction kinetics of Headwater fly ash leached in 7.5 M KOH at different temperatures.

FIG. 13 illustrates the analysis of reaction kinetics of BSI fly ash leached in 7.5 M KOH at different temperatures.

FIG. 14 illustrates the analysis of reaction kinetics of six different fly ashes leached in 7.5 M KOH at 75° C.

FIG. 15 shows the relative mass of gel (β′) produced as a function of reaction. progress (α); data include results from all six fly ashes and temperatures; 5, 7.5, and 10 M KOH.

FIG. 16 shows the relative mass of fly ash dissolved (α′) as a function of reaction progress (α); data include results from all six fly ashes and temperatures; 5, 7.5, and 10 M KOH.

FIG. 17 illustrates the dependence of glass network dissolution process on network former concentration in 7.5 M KOH.

FIG. 18 shows the compressive strength of geopolymers as a function of reaction progress (Headwater fly ash).

### DETAILED DESCRIPTION OF PREFERRED EMBODIMENTS

### Materials and Methods

The properties of six fly ash samples from six different power plants in the USA were investigated. These samples represent fly ashes low in calcium (class F fly ash). The compositions of the fly ash samples were analyzed by X-ray fluorescence spectroscopy (XRF). A series of NIST (National Institute of Standards and Technology) reference materials were used for instrument calibration. The loss on ignition (LOI) was measured for all samples following the ASTM D7348-08 procedure. The composition of a fly ash sample was calculated by normalizing the XRF data to 100 wt %, taking LOI into account. The size of the fly ash particles was determined with a laser particle size analyzer.

All fly ash samples were analyzed quantitatively for crystalline phases using X-ray powder diffraction (XRD). Phase identification was accomplished by comparing diffraction patterns with JPDCS files. Approximately 5 g of fly ash was used for each measurement. Following a quantitative powder diffraction analysis procedure [47], 2 wt % of rutile was added to each sample as an internal standard. The mass fraction of glass was determined by difference after correction for LOI. The chemical composition of the glass phase was calculated by subtracting LOI and the composition of the crystalline phases from the overall composition.

The fraction of glass phase in a fly ash sample was determined by a method described by Fernandez-Jimenez et al. [48]. This method prescribes treatment of fly ash with 1 wt % hydrofluoric acid solution for 6 hours to dissolve the glass phase under constant stirring, which leaves the crystalline phases (e.g., mullite, quartz, carbon, hematite and magnetite) unaffected. However, Fernandez-Jimenez et al. [48] reported that a second treatment might be needed to obtain correct glass contents for fly ashes with high glass contents. In the present case, constant weight of fly ash residues was not obtained until after the fourth treatment. The total duration of the experiment was 24 hours. The fly ash samples were dried in an oven at 105° C. before and after treatment in hydrofluoric acid until constant weight was attained. In Fernandez-Jimenez's procedure, samples are heated to 1000° C. after leaching in hydrofluoric acid, which can be applied only to fly ash samples with a content of unburned carbon low enough not to affect the result of HF leaching. Some fly ashes have a fairly high content of carbon (Table 1), which upon heating to 1000° C. in air would burn and obscure the measurement of the glass content.

### Measuring Reactivity

In this work, fly ashes were characterized by their reactivity, measured in terms of the reaction progress (α), which is the relative mass of reacted glass in alkali hydroxide solution as a function of time. Typically, the glass phase is the only reactive component of fly ash in an alkaline solution below 100° C. Consequently, reactivity depends on the glass composition—i.e., it is inversely proportional to the chemical durability of the glass. Hence, reaction progress depends on the molarity and kind of alkali (e.g., Li, Na, K, Ca, etc), temperature, and on the water-to-solid-ratio (W/S). The alkaline solution can be a solution comprising at least one, such as at least 2, at least 3, etc., alkaline ions. The term ‘reactivity’ therein applies to fly ash in its entirety, not only to the glass phase; for this reason, the reaction progress may not reach 100% in the presence of less reactive or inert constituents in a fly ash sample.

α was measured in two ways: (1) directly by mass loss and (2) indirectly by solution analysis. If mₒ is the mass of fly ash initially and mₐₗₖ is the residual mass after leaching in alkali hydroxide solution, then the relative mass loss α′ of the fly ash is

α=(mₒ−mₐₗₖ)/mₒ=1−mₐₗₖ/mₒ. (1)

If the molarity of the alkali and the water-to-solid-ratio W/S are constant, then mₐₗₖ, is only a function of temperature (T). Because frequently a surface layer (gel) forms on the glass surface, leached fly ash sample was immersed in hydrochloric acid (HCl) to dissolve the gel. Under these conditions silica is assumed to be present in colloidal form, since SiO₂ is insoluble in HCl and a precipitate did not form. The gel is thus considered as reacted glass. The mass dissolved in acid originates from reacted but undissolved glass, and potentially from additional material, such as water, hydroxyl groups, and alkali ions. Hence, if α′ in Eq. (1) is determined by a mass loss measurement, the mark must be corrected for the additional material, δ, in the gel:

α′_{corr}=(mₒ−mₐₗₖ+δ)/mₒ=α′+δ/mₒ. (2)

The mass δ depends on the temperature. The mass fraction α′ can also be measured by analyzing the alkaline leachate for all glass constituents and recalculating them as oxides.

At higher reaction progress, crystals may form within or on top of the gel layer. Such new crystalline phases were considered as a part of the reacted fly ash (i.e., essentially reacted glass) because they contain fly ash constituents that otherwise would not be accounted for. However, if one or more crystalline species contain constituents from the leachant (i.e., the alkali ion, hydroxyl groups and/or water), this treatment may introduce an error. The error is small as long as the mass of crystals in the gel is small compared with the mass of the gel. The relative mass of gel and/or new crystals is β′:

β′=(mₐₗₖ−m_{ac})/mₒ, (3)

where m_{ac} is the residual mass of fly ash after treatment in acid. The mass m_{ac} depends on temperature indirectly because δ varies with temperature. If β′ is determined by mass loss measurement, a correction δ should be applied to m_{ac} to represent the fraction of fly ash converted into gel and crystals:

β′_{corr}=(mₐₗₖ−m_{ac}−δ)/mₒ=β′−δ/mₒ. (4)

β′_{corr} can also be calculated from analytical data, if the solution containing the dissolved gel and crystals is analyzed for all its constituents. If δ/mₒ<<β′, β′_{corr}≈β′—i.e., there should not be a significant difference between the results from solution analyses and mass loss measurements. Finally, the reaction progress α is given by:

α=α′_{corr}+β′_{corr}=(mₒ−m_{ac})/mₒ. (5)

Reactivity is dα/dt—i.e., the rate at which the reaction progresses between a fly ash and an alkali hydroxide solution under various experimental conditions. In this sense, the fly ash with the highest reaction progress at a given time has the highest reactivity under a given set of experimental conditions.

Eq. (5) shows that the reaction progress of a fly ash can either be determined by solution analyses or by measuring the residual mass after exposure of the leached fly ash to HCl.

In general, mass loss measurements may be preferred versus solution analyses because they are faster and easier to perform. However, if the objective is to research details of the leaching process, which includes determination of quantities such as α′, β′ and δ, use of both methods is preferable, because they complement each other. Mass loss measurements are more precise than solution analyses, but the calculated quantity may not be accurate. Certain corrections apply (Eqs. (2) and (4)). The parameter δ can be quantified by comparing analytical and mass loss measurements. Solution analyses yield accurate quantities provided that the alkali used for leaching does not occur in significant concentration in the fly ash. For example, if a fly ash contains a significant amount of potassium and the leachant is a concentrated solution of KOH, then potassium from the fly ash cannot be determined. Solution analyses are less precise because all dissolved fly ash constituents must be analyzed quantitatively and summed as oxides to calculate a mass loss.

### Leaching Experiments

Leaching experiments were conducted with all six fly ash samples in a water bath equipped with a vibrating system, except at room temperature (20° C.). The vibration system helped prevent fly ash particles from agglomerating. Potassium hydroxide was used because all previous work was conducted with KOH during research and development of geopolymer based waste forms for radioactive and hazardous waste streams [2]. Other alkaline solutions can be used as well. For example, the solution can comprise any alkali metal or alkali earth cation and other suitable anions. The molarities of the alkaline solution can be for example, between about 1 and about 20 M, such as between about 2 and about 15, such as between about 5 and about 10 M. In some embodiments, the molarities of the alkaline solution were 1, 3, 5, 7.5, and 10 M, the reaction time was up to 336 hours (14 days), and the temperatures were 20, 35, 40, 50, 60, and 75° C. The fly ash was dried at 105° C. for three hours. A mass of 2.5 g fly ash was mixed with the alkaline solution in polyethylene bottles. The alkaline solution contained 100 ml water and alkali hydroxide of various amounts. Before mixing with the fly ash, thermal equilibrium of the alkaline solution was attained at a designated temperature in the polyethylene bottle. Calibrated thermocouples were used for temperature measurements. Temperatures fluctuated by not more than ±1° C. A water-to-fly-ash ratio of W/S=40 was used and this ratio was kept constant in all experiments. A water-to-solid-ratio typical of startup mixtures for geopolymers would range from 0.2 to 0.5 (i.e., much less water would be used). The W/S ratio is discussed further below.

After leaching, the suspension was filtered through a 0.6 μm polycarbonate membrane. The residue was washed with deionized water three times and then put in absolute ethanol for three hours to stop further hydration of the glass phase. Then the residue was dried at 105° C. for three hours. The leachate was chemically analyzed.

The dried fly ash residue was immersed in diluted (1:20) HCl at room temperature for 3 hours under slow stirring to dissolve any reaction products generated by the preceding leaching process. About 0.5 g of the leached fly ash was put into a polyethylene bottle containing 250 ml diluted HCl. The mixture was stirred with a magnetic stirrer for three hours. Then the suspension was filtered through a 0.6 μm polycarbonate membrane and washed with deionized water until neutral. The residue was dried at 105° C. for three hours and weighed. Finally, α was calculated according to Eq. (5).

In addition, each of the six pristine fly ash samples was subjected to chemical attack in 1:20 diluted HCl at room temperature for three hours under slow stirring without previous leaching in KOH. The mass loss after acid attack was less than 2 wt %. The presence of soluble salts, fly ash particles smaller than 0.6 μm, and probably leaching of a small amount of the vitreous phase may explain this mass loss. Since this mass loss was relatively small, it was used to make corrections to leach data only for the lowest reaction progress data, specifically those measured at temperatures up to 50° C. after the shortest reaction time.

### Analysis of Leached Products

Direct current plasma atomic emission spectroscopy (DCP-AES) was used to measure concentrations of elements coming from alkaline and acidic leaching of the fly ashes. The spectrometer was calibrated using NIST traceable standard solutions. The relative error for DCP-AES analyses was ±3% or less. The elements analyzed were Si, Al, Fe, Li, B, Na, Mg, Ca, Ti, Ba, Cr, Mn, Ni, Sr, P, and Zr.

Leached fly ash samples were also analyzed quantitatively for newly formed crystalline phases using X-ray powder diffraction (XRD). Pristine fly ash particles and particles leached in alkaline solution were investigated using a scanning electron microscope, equipped with an energy-dispersive X-ray spectroscopy device. To prepare the samples, the pristine or leached fly ash particles were first embedded in epoxy. The specimens were polished using diamond papers from 30 μm to 0.5 μm grain size and absolute ethanol as lubricant. The specimens were used to study the microstructure of fly ash particles before and after leaching. Pristine or leached fly ash particles were also deposited on a conductive tape. The specimens were coated with carbon. Secondary electron imaging (SEI), backscattering electron imaging (BEI), and energy-dispersive X-ray spectroscopy (EDS) were used to characterize pristine and leached fly ash samples.

### Results

The compositions of the six fly ash samples were reported below. The reactivity was characterized by its dependence on glass composition, molarity of KOH, temperature, appearance of new crystalline material, morphology of particles after leaching, the composition of the leachates, and the composition of gel layers, which formed on the glass surface during leaching in alkaline solution.

### Fly Ash Composition

The chemical compositions of the fly ashes are shown in Table 1. As shown therein, the main constituents of the glass phase are SiO₂, Al₂O₃, and Fe₂O₃. The content of these three oxides together is 77 to 81 wt %, depending on the fly ash. Other oxides in the glass phase include K₂O (2.1-2.5 wt %), TiO₂ (1.3-2.1 wt %), CaO (1.0-3.3 wt %), MgO (0.7-0.9 wt %), and SO₃ (0-1.3 wt %) and some trace elements. Table 2 shows the compositions of the glass and the crystalline phases separately. The mass fraction of the glass phase in the fly ashes range between 69 and 80 wt % after subtraction of LOI from the total mass. The mass fraction determined indirectly by X-ray analysis agrees very well with that measured by dissolution in hydrofluoric acid.

TABLE 1
Compositions of six fly ashes (as received)

| | Power plant | | | | | |
|---|---|---|---|---|---|---|
| | | | Brandon | Head- | New- | Chalk |
| | BSI | Wagner | Shores | water | burgh | Point |
| | Abbreviated | | | | | |
| | BSI | WAN | BRS | HW | NEW | CHP |
| Constituents | Weight percent | | | | | |
| SiO₂ | 52.6 | 52.5 | 51.4 | 48.4 | 47.4 | 50.8 |
| Al₂O₃, | 24.8 | 25.3 | 25.2 | 25.0 | 24.8 | 25.6 |
| Fe₂O₃ | 3.4 | 4.4 | 2.8 | 8.0 | 11.4 | 12.7 |
| MgO | 0.7 | 0.7 | 0.6 | 0.9 | 0.9 | 0.8 |
| CaO | 1.0 | 1.1 | 1.0 | 1.4 | 3.3 | 1.0 |
| SrO | 0.1 | 0.1 | <0.1 | 0.1 | 0.2 | 0.1 |
| BaO | <0.1 | <0.1 | <0.1 | 0.1 | 0.1 | 0.1 |
| Na₂O | <0.1 | 0.2 | <0.1 | 0.4 | 0.5 | <0.1 |
| K₂O | 2.5 | 2.4 | 2.2 | 2.6 | 2.2 | 2.5 |
| P₂O₅ | 0.2 | 0.2 | 0.2 | 0.5 | 0.9 | 0.2 |
| SO₃ | <0.1 | 0.8 | 0.5 | 1.2 | 1.3 | 0.9 |
| TiO₂ | 1.9 | 1.8 | 2.1 | 1.5 | 1.4 | 1.6 |
| Other* | 0.2 | 0.3 | 0.2 | 0.4 | 0.4 | 0.3 |
| LOI | 12.6 | 10.3 | 13.7 | 9.6 | 5.4 | 3.3 |
| Total | 100.0 | 100.0 | 100.0 | 100.0 | 100.0 | 100.0 |
| Particle size | 30.6% | 37.0% | 32.8% | 26.8% | 18.2% | 77.7% |
| >45 μm | | | | | | |

*Comprises oxides of As, Cr, Cu, Ga, Mn, Ni, Zn, Zr, and rare-earth elements

About 80% of the particles sizes in HW fly ash range between 10 and 100 μm with an average of 44 μm. The biggest particles frequently formed clusters of smaller particles. The glass phase inside the fly ash particles was spherical in shape. Other fly ashes showed similar results.

The mass fractions of crystalline phases range from 15 to 21.8 wt % (Table 2). The crystalline phases are mullite (7.3-13.4 wt %) and quartz (2-5.3 wt %). In addition, three fly ashes (HW, NEW, CHP) contain magnetite. XRD data suggested that the iron phase could be either magnetite or maghemite. The presence of magnetite was supported by Mossbauer Spectroscopy.

TABLE 2
Composition of the glass and crystal phases
in fly ash samples (as received)

| | | Power plant | | | | | |
|---|---|---|---|---|---|---|---|
| | | | | Shores | Head- | | Chalk |
| | | BSI | Wagner | Brandon | water | Newburgh | Point |
| | | Abbreviated | | | | | |
| | | BSI | WAN | BRS | HW | NEW | CHP |
| Constituents | | Weight percent | | | | | |
| Glass | Al₂O₃ | 16.1 | 15.8 | 15.5 | 19.8 | 15.2 | 19.3 |
| | SiO₂ | 46.3 | 43.5 | 44.3 | 43.9 | 41.6 | 43.8 |
| | Fe₂O₃ | 3.4 | 4.4 | 2.8 | 4.2 | 5.0 | 9.4 |
| | CaO | 1.0 | 1.1 | 1.0 | 1.4 | 3.3 | 1.0 |
| | MgO | 0.7 | 0.7 | 0.6 | 0.9 | 0.9 | 0.8 |
| | BaO | <0.1 | <0.1 | <0.1 | 0.1 | 0.1 | 0.1 |
| | K₂O | 2.5 | 2.4 | 2.2 | 2.6 | 2.2 | 2.5 |
| | Na₂O | <0.1 | 0.2 | <0.1 | 0.4 | 0.5 | <0.1 |
| | P₂O₅ | 0.2 | 0.2 | 0.2 | 0.5 | 0.9 | 0.2 |
| | SO₃ | <0.1 | 0.8 | 0.5 | 1.2 | 1.3 | 0.9 |
| | SrO | 0.1 | 0.1 | <0.1 | 0.1 | 0.2 | 0.1 |
| | TiO₂ | 1.9 | 1.8 | 2.1 | 1.5 | 1.4 | 1.6 |
| | Other* | 0.2 | 0.3 | 0.2 | 0.4 | 0.4 | 0.3 |
| | Subtotal | 72.4 | 71.2 | 69.5 | 77.0 | 72.8 | 80.0 |
| | glass | | | | | | |

| | | | | | | |
|---|---|---|---|---|---|---|
| LOI | 12.6 | 10.3 | 13.7 | 9.6 | 5.4 | 3.3 |
| Quartz | 2.8 | 5.3 | 3.4 | 2.4 | 2.0 | 4.6 |
| Mullite | 12.2 | 13.2 | 13.4 | 7.3 | 13.4 | 8.8 |
| Magnetite | <0.1 | <0.1 | <0.1 | 3.7 | 6.4 | 3.3 |
| Total | 100.0 | 100.0 | 100.0 | 100.0 | 100.0 | 100.0 |

*Comprises oxides of As, Cr, Cu, Ga, Mn, Ni, Zn, Zr, and rare-earth elements

The fly ash samples contained a significant amount of unburned porous carbon. The loss on ignition (LOI) was attributed to carbon (Table 2), neglecting contributions from minute amounts of carbonate and hydroxyl that may be present. The unburned carbon was assumed not to have reacted with KOH.

The glass phases in the six fly ash samples are similar in chemical composition. Silica concentrations (reactive silica) range from 41.6 to 46.3 wt % and alumina concentrations (reactive alumina) from 15.2 to 19.8 wt %. Silica and alumina in the glass phase of fly ash are the main constituents participating in pozzolanic reactions in geopolymer precursor mixtures. Silica contents higher than 20 wt % are considered desirable for a fly ash to be a reactive constituent in cement and geopolymer materials.

### Reactivity

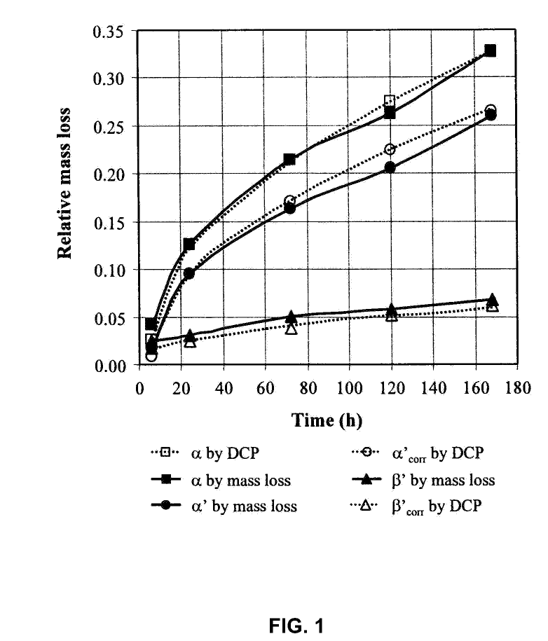

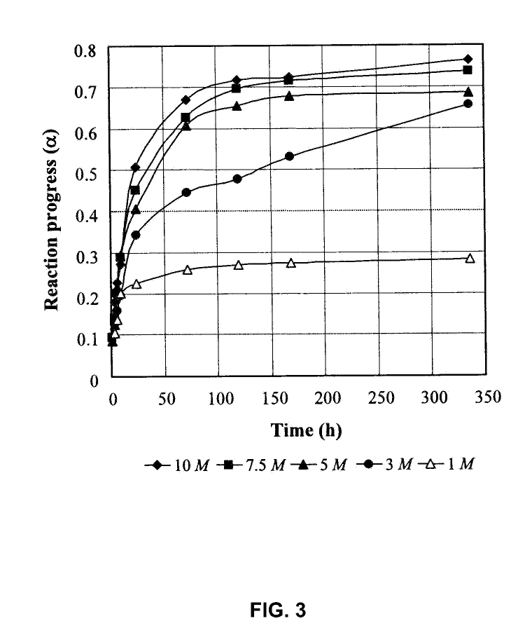

FIG. 1 shows the result of mass loss measurements on fly ash. In this case, Headwater (HW) fly ash was leached in 7.5 M KOH for two weeks at 40° C. The two uppermost curves show the reaction progress α (Eq. (5)), determined either by direct mass loss measurement (the solid line) or calculated based on DCP-AES solution analyses (the dotted line). The agreement of the results is within the limits of experimental errors. The solid curve in the middle of FIG. 1 shows the relative mass loss α′ (Eq. (1)) after leaching HW fly ash in 7.5 M KOH solution. The respective dotted curve shows α′_{corr} (Eq. (2)), which was calculated based on DCP-AES analyses of the leachates. Values of α′_{corr} are slightly higher than respective values of α′ because they include the correction δ (Eq. (2)). The relative mass loss β′ (Eq. (3)) is shown by the solid curve on the bottom of FIG. 1. β′ was determined by measuring masses of fly ash samples after dissolving the gel layer on the glass surface in HCl (Eq. (3)). The dotted curve shows the respective relative mass loss β′_{corr} (Eq. (4)), based on chemical analyses of the acid leachates. Eqs. (3) and (4) suggest that the dotted curve on the bottom of FIG. 3 runs below the respective solid curve, whereas Eqs. (1) and (2) suggest that the dotted curve α′_{corr} runs above the respective solid curve. This is in qualitative agreement with the experimental results. However, the differences between the respective data are too small to be significant. The discrepancies show that a measurable amount of hydroxyl groups and alkali metal ions are contained in the gel. FIG. 1 shows also that δ is relatively small in the measured range of α<0.35. As shown below, δ becomes more important at higher reaction progress.

### Effect of Temperature, Molarity of KOH, and Glass Composition

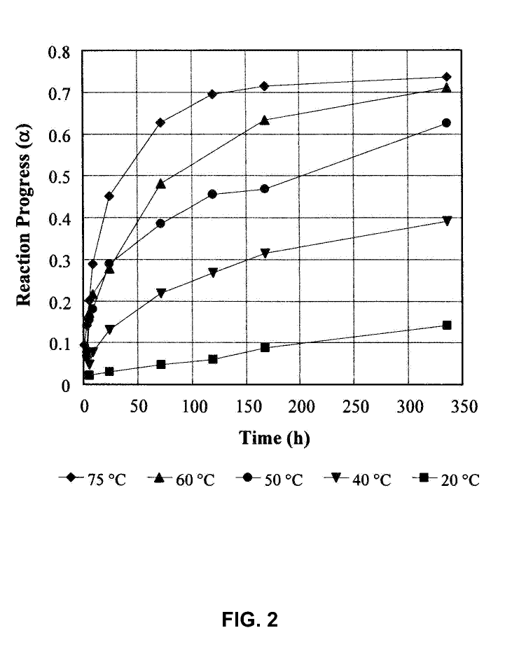

FIG. 2 shows the effect of temperature on reaction progress in 7.5 M KOH solution for HW fly ash. Qualitatively, this temperature dependence is typical of all six fly ashes. Reaction progress increases with increasing temperature. The temperature can be of any value, depending on the application and the other testing parameters. For example, the temperature can range from room temperature to about 100° C., such as between about 30° C. to about 90° C., such as about 40° C. to about 80° C. In some embodiments, the rate of reaction tends to decrease with time. At 60° C. and 75° C. reaction progress reaches its highest possible value after two weeks. Thus, in one embodiment, when an accelerated testing condition is desired, the temperature can be set to about 75° C. The glass phase has reacted completely with KOH. As explained previously, the quantity α does not reach the value of α=1 because α refers to fly ash as a whole, not only to the glass phase. Evidently, the crystalline phases (mullite, quartz, and magnetite) and unburned carbon behave practically like inert materials.

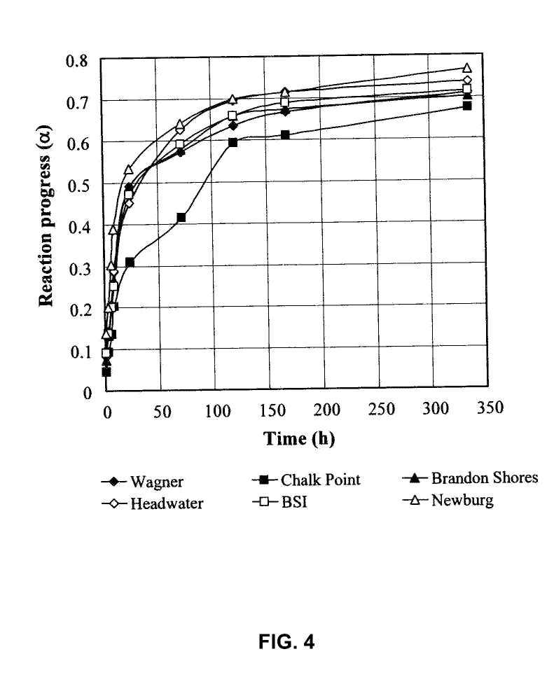

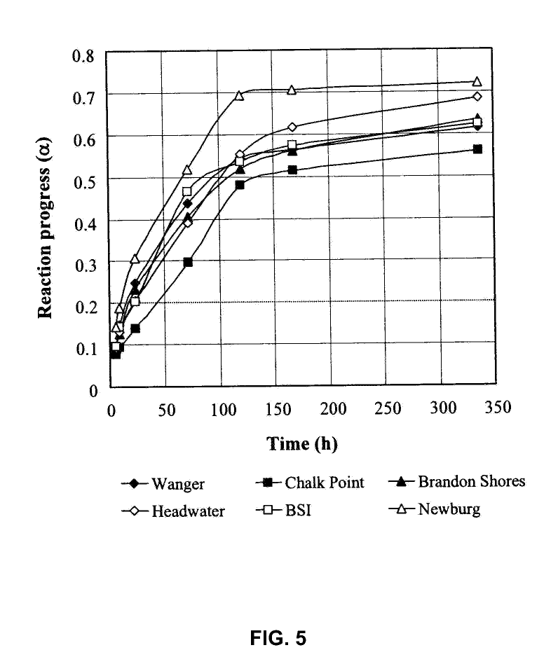

FIG. 3 shows the reaction progress of HW fly ash as a function of time at different KOH molarities at 75° C. As expected, reaction progress increased with increasing OH⁻ molarity. The effect of an increase in OH⁻ molarity is relatively small above 5 M. The rate of reaction decreases with time. Except in 1 M KOH solution α reaches or approaches highest values of 0.7 to 0.8, which correspond to the mass fractions of glass in the present fly ashes (Table 2, line subtotal). SEM micrographs confirmed that very little glass phase was left after two weeks at high KOH molarity. FIGS. 4 and 5 show reaction progress results of all six fly ashes in 7.5 M KOH at 75° C. and 50° C., respectively. While different fly ashes show different reactivity, two general observations were applicable to all six fly ashes: (1) at 75° C. and at KOH concentrations >5 M, reactivity of all six fly ashes was similar; (2) at lower temperatures but still at KOH concentrations >5 M, different fly ashes became more distinguishable.

### Crystallization

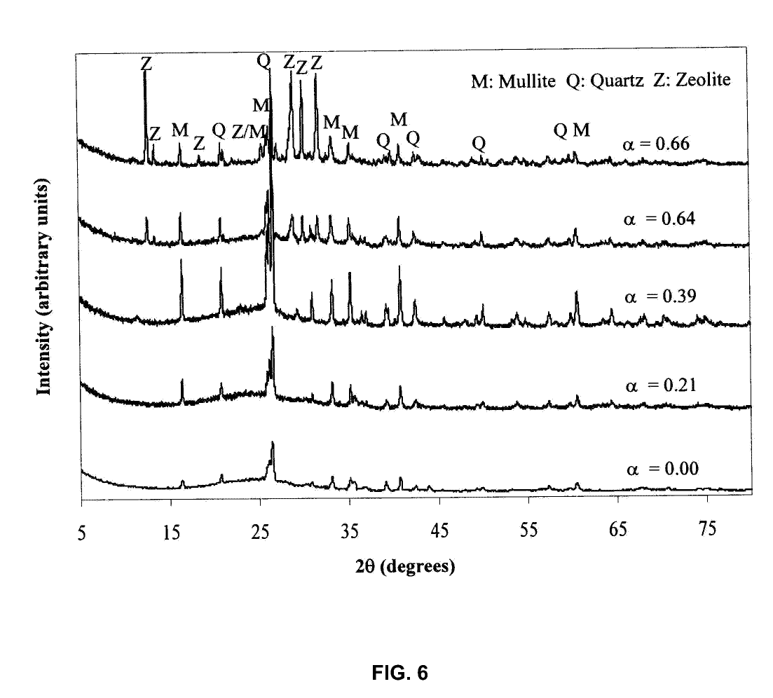

FIG. 6 shows X-ray diffraction patterns of HW fly ash leached in 7.5 M KOH solution at 50° C. Mullite and quartz are phases present in the pristine fly ash (α=0). All fly ash patterns in FIG. 6 show a halo between 20°<2θ<35°, indicating the presence of amorphous material (i.e., glass in the pristine fly ash and glass and gel in leached samples). As the reaction progress increases from zero to 0.66, an additional crystalline phase (marked Z) appears. This phase was identified as Linde F zeolite (KAlSiO₄.1.5H₂O, JCPDS 38-0216). The yield of zeolite increased strongly with increasing reaction progress (compare α=0.64 and α=0.66 in FIG. 6). The pattern for α=0.39 was measured with increased sensitivity, compared to the other patterns. There was no evidence of zeolite yet. A more detailed XRD study with all fly ashes at different leaching conditions showed that: (1) Linde F zeolite was the only new phase, (2) Linde F zeolite was observed at temperatures as low as 35° C. and α>0.45, and (3) the KOH concentration had to be >3 M for the zeolite to crystallize. Linde F zeolite was not observed at 20° C. because the highest reaction progress was 0.14 in 7.5 M KOH, far less than 0.45. The reaction rate at 20° C. was very small. It was observed that diluted HCl (1:20) dissolved both the gel and the zeolite quantitatively.

### Morphology of Leached Fly Ash

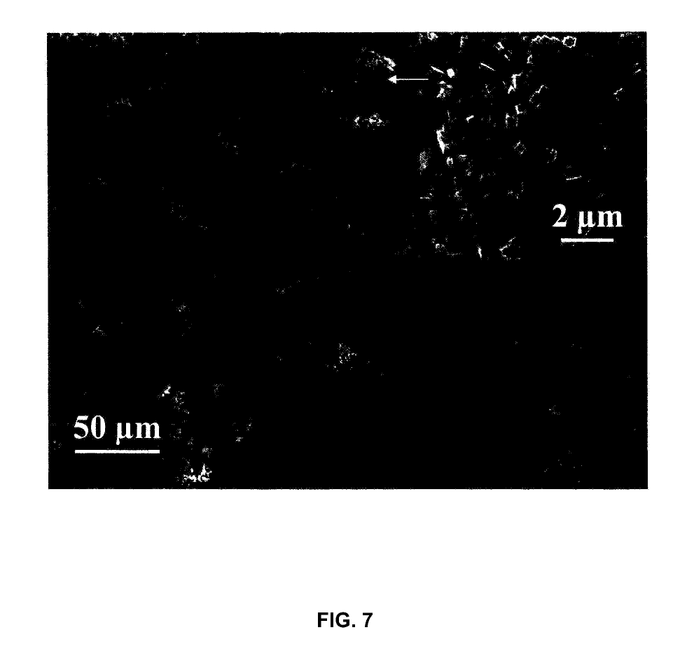

FIG. 7 shows a SEI micrograph of HW fly ash leached in 7.5 M KOH at 75° C. for 72 hours (α=0.62). The spherical particles are glass particles covered with the reaction products. The inlay in FIG. 7 shows the surface of a leached glass particle identified by the arrow. At a higher magnification of the inlay, individual crystals of the Linde F zeolite become visible. The prismatic morphology is consistent with that reported by Sherman [49] for Linde F zeolite, and energy-dispersive X-ray spectroscopy (EDS) analysis confirmed its composition.

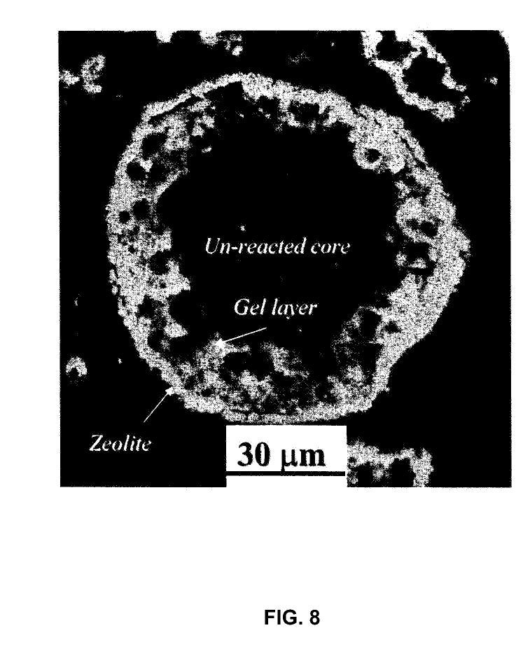

FIG. 8 shows a SEI micrograph of a glass particle of HW fly ash after leaching in 7.5 M KOH at 75° C. for 72 hours (α=0.62). The particle consists of an unreacted core of glass, a gel layer, and an outer crust of zeolite. The important detail here is that the zeolite was found only in the crust. There were no crystals inside the gel. In one embodiment, the gel layer and/or the core is substantially free, such as completely free, of a crystal, such as a zeolite crystal. FIGS. 7 and 8 are typical of all fly ash samples leached under the aforementioned conditions.

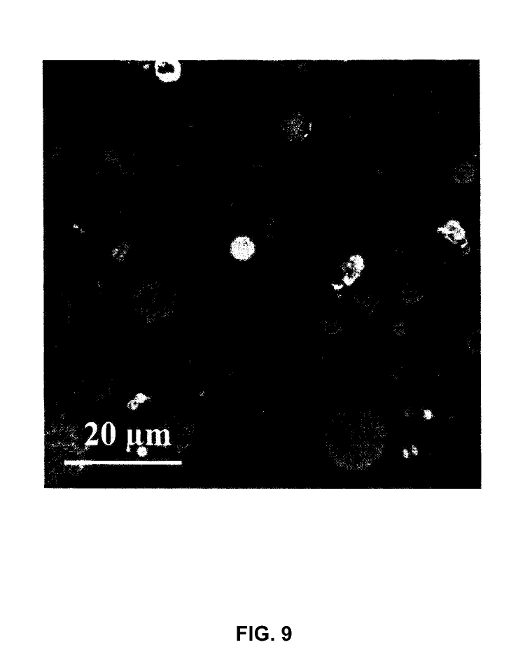

FIG. 9 shows the microstructure (BEI micrograph) of leached HW fly ash particles with on average 6% of the glass phase left (7.5 M KOH, 75° C., α=0.71; initial glass content 77%). In some embodiments, the percentage of the glass phase can be less than about 20%, such as less than about 10%, such as less than about 5%, such as less than about 2%. The zeolite crust is partially missing because some crystals were detached during sample preparation. Beneath the zeolite crust can be a thin gel layer (less than 1 μm thick), which can be rich in higher Z elements, such as iron (Fe₂O₃˜26 wt %) and alkali-earth elements (CaO˜8 wt %, MgO 2 wt %, and some TiO₂ and ZnO). The gel layer can comprise other types of metal oxides as well. The gel layer can also comprise a non-metal oxide, such as silica. In one embodiment, the layer comprises SiO₂ and Al₂O₃. It is not yet clear whether iron is present as goethite (FeO(OH)) or hematite (Fe₂O₃) or whether it is a part of the aluminosilicate gel. Between the bright thin layer and the unreacted core of a fly ash glass particle is a darker gel phase with a thickness about 1-5 μm. The less bright gel layer contains more SiO₂ and Al₂O₃ and much less Fe₂O₃ than the thin layer on top. The bright cores of thickest glass particles consist of unreacted glass.

### Results of Leachate Analyses

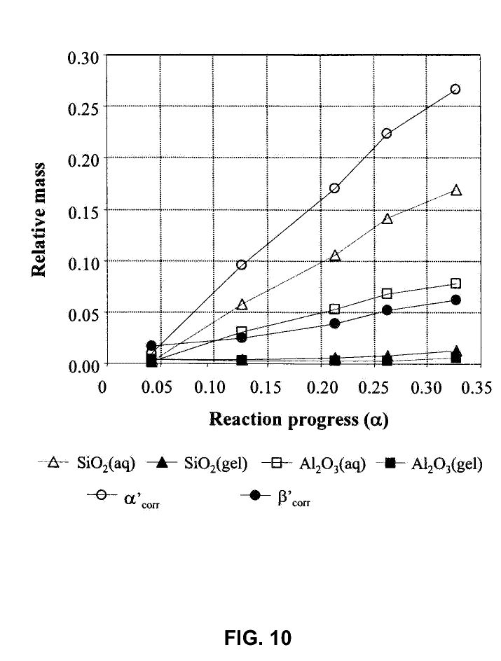

FIG. 10 shows the partitioning of two glass constituents SiO₂ and Al₂O₃ between the aqueous phase and the gel layer after leaching HW fly ash in 7.5 M KOH at 40° C. The gel was dissolved in HCl and analyzed by DCP-AES. The partitioning of SiO₂ and Al₂O₃ was illustrated by plotting relative masses of oxides (g/g-fly ash) versus reaction progress. Since practically no other constituents of the glass were found in the leachate, the sum of SiO₂ and Al₂O₃ is the total mass of glass dissolved (open circles in FIG. 10). The mass of SiO₂ and Al₂O₃ found in the gel layer is relatively small and does not add up to the total mass of gel (filled circles in FIG. 10). Analysis of the dissolved gels showed that Fe₂O₃, and to a lesser extent CaO, MgO, and TiO₂, make up the difference. Small amounts of Sr, Ba, Ni, and Mn were also detected in the gel. At values of α<0.1, Ca was found in the aqueous phase, but Ca precipitated at higher reaction progress and became a part of the gel. This finding is consistent with results reported by Lee and van Deventer [43], who studied the dissolution of Gladstone fly ash (class F) in KOH solution. The overall finding described herein is that most (80%) of the reacted glass was dissolved, up to α=0.33 (FIG. 10). Only about 20% of the reacted glass was found in the form of gel on the surface of the residual glass.

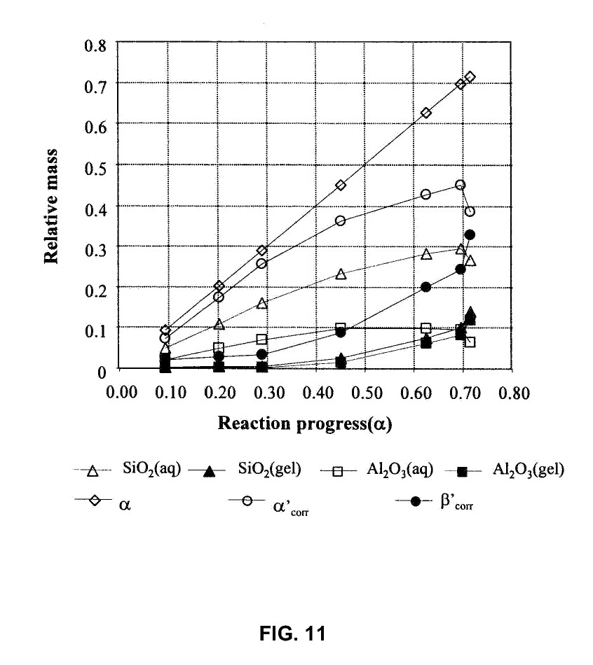

FIG. 11 shows mass fractions (g/g-fly ash) of dissolved SiO₂ (open triangles) and Al₂O₃ (open squares) measured by DCP-AES solution analyses as a function of the reaction progress for HW fly ash leached in 7.5M KOH at 75° C. Other elements leached from the fly ash were neglected because their concentrations were miniscule in the leachates. Summation of SiO₂ and Al₂O₃ yielded α′_{corr} (Eq. (2))—i.e., the relative mass loss of the fly ash after leaching. FIG. 11 also shows relative masses of SiO₂ (solid triangles) and Al₂O₃ (solid squares) in the gel. Summing these oxides together with other glass constituents (Fe₂O₃, CaO, MgO, TiO₂—not shown) yielded β′_{corr} (Eq. (4))—i.e., the relative mass of reacted fly ash retained as gel on the surface of the residual glass particles. Summation of α′_{corr} and β′_{corr} yielded α, the total reaction progress (Eq. (5)).

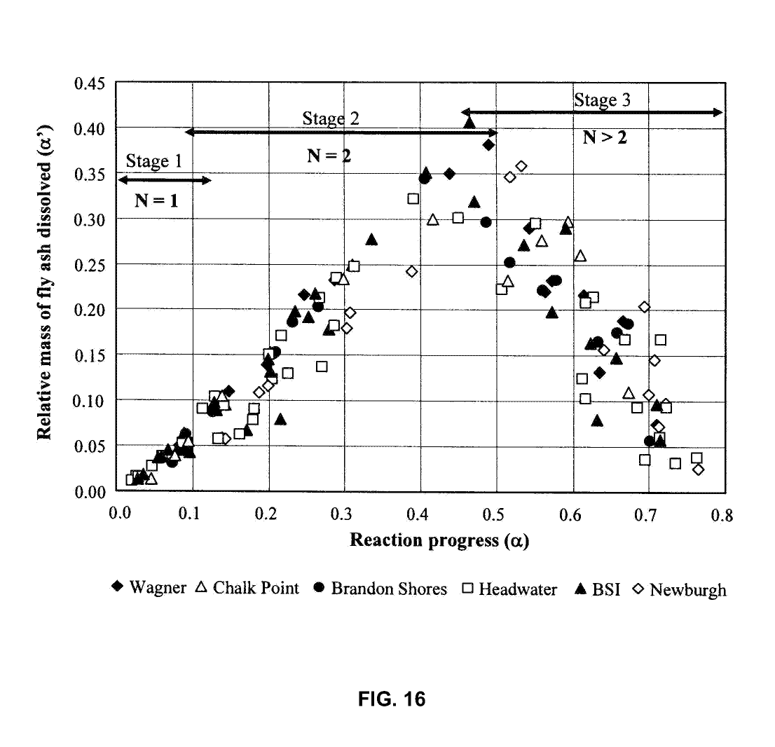

The behavior of the major oxides was the same at 40° C. and 75° C. as long as α was <0.33. The contribution of gel to the total reaction progress increased to almost 50% at higher α values, reaching 0.34 g/g-fly ash (β′_{corr}) compared with 0.38 g/g-fly ash dissolved (α′_{corr}) at α≈0.71. In comparison, the relative mass of gel β′ (Eq. (3)) determined by a mass loss measurement was 0.654 g/g-fly ash at α≈0.71. Thus, the δ value in equations (2) and (4) was 0.316 g/g-fly ash. Consequently, δ is not negligible at higher values of α. The significance of this finding will be discussed later in context with FIG. 16.

In this study, a water-to-solid (W/S) ratio of 40:1 was used, which ratio is roughly 100 times higher than that in geopolymer pastes. Depending on the application, other ratios can also be used. In some embodiments, the ratio can be greater than or equal to about 1 but less than or equal to 1000, such as between about 5 and about 800, such as between about 10 and about 500, such as between about 20 and about 200. For example, the ratio can be at least about 30, such as at least about 35, such as at least about 45, such as at least about 50. The maximum achievable reaction progress α (i.e., digestion of the glass phase in fly ash) depends on W/S. For example, at a ratio of 0.35:1, α is up to 50% less than at 40:1. However, the rates at which the digestion of the glass phase progresses in 10 M KOH at 75° C. are similar at 40:1 and 0.35:1. Most of the glass is consumed within a day or two. This was found in some tests conducted at 0.35:1. At low W/S, reactions of the glass phase in fly ash are more difficult to study because almost complete consumption of water makes analysis of residual liquid (the leachate) extremely difficult. One working hypothesis herein was that studying the reactivity of fly ash at a relatively high W/S ratio may provide insight into what is happening at much lower W/S ratios, i.e. under conditions needed to make geopolymers.

First, the results above are interpreted with the help of a reaction kinetics model describing a particular type of reaction and translating it into a rate equation. Khawam and Flanagan [50] published a detailed review of processes, models, and rate laws governing solid-state reactions. After consideration of various models and an analysis of the data, a rate equation first published by Jander [51] Eq. (6) was adopted:

[1−(1−α)^{1/3}]²=K₂·t, (6)

where K₂=k₂/r², and k₂ has the dimensions of a diffusion coefficient; r is the radius of a sphere [50]. Jander's model provides a rate equation applicable to a heterogeneous system (solid/liquid), if the reaction takes place beneath a surface layer and the reaction rate is controlled by transport (diffusion) of species towards or away from the front of the reaction. The reaction itself is not rate limiting.

Kondo et al. [52] modified the Jander equation for a broader application by introducing a reaction grade N, Eq. (7):

[1-(1−α)^{1/3}]^{N}=K_{N}·t, (7)

or in linear form, Eq. (8)

1n[1−1(1−α)^{1/3}]=1/N·1n(K_{N})+1/N·1n(t), (8)

where K_{N} is the rate constant replacing K₂. The advantage of a variable N is that Eq. (8) can be used to model consecutive sometimes overlapping processes. Following Kondo et al. [52], N has the following meaning:
1) If a process is controlled by a chemical reaction occurring at the surface of a spherical particle, or by dissolution of reactants or the precipitation of reaction products, then N≦1. This is the ‘geometrical contracting sphere/cube model’ derived by KhawaM and Flanagan [50].
2) If the reaction is controlled by diffusion of reactants through a porous layer of reaction products, then 1<N<2. This is the diffusion model derived by KhawaM and Flanagan [50].
3) If the reaction is controlled by diffusion of reactants through a dense layer of reaction products, then N may be >2.

Shi and Day [38] used Eq. (7) to model reactions of an activator (Na₂SO₄ and CaCl₂) in a lime-pozzolan blend. Dabic et al. [53] and Krstulovic and Dabic [54], modeled cement hydration. Cabrera and Rojas [55] modeled hydration of the metakaolin-lime-water system.

The glass particles in fly ash are spherical in a first approximation. FIGS. 7 to 9 support this approximation. The glass phase forms at high temperature when the viscosity of the glass is relatively low and surface tension high enough to form spherical particles. The only mismatch with respect to the Jander equation is that the glass particles occur in different sizes.

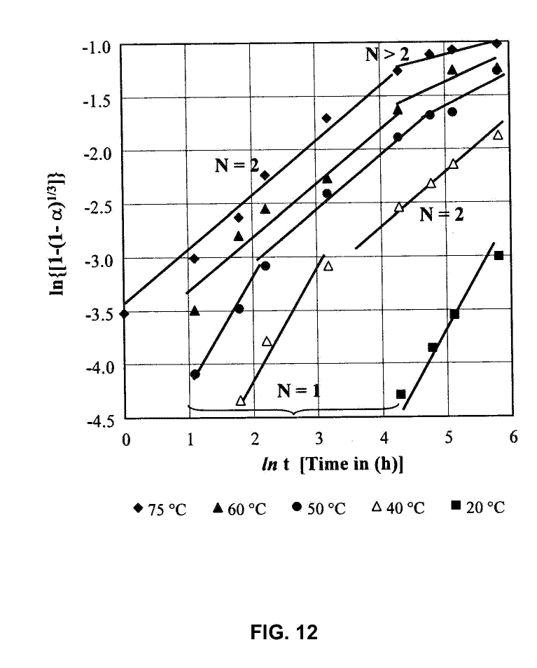

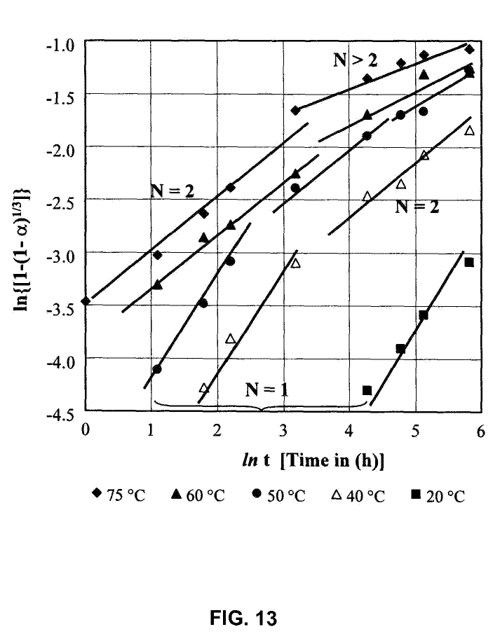

The modified Jander model (Eq. (8)) was applied to the data to obtain the following results: FIGS. 12 and 13 show plots of Eq. (8) fitted to reaction progress data measured for HW and BSI fly ash, respectively. Both fly ashes were leached in 7.5 M KOH solutions at various temperatures. The slopes of the straight lines are the inverse of Kondo et al.'s reaction grade N [52]. At the lowest temperature (20° C.), only one curve with a slope of 1/N=1 was obtained for data up to 336 hours, i.e., 14 days (filled squares). Experiments were conducted for no longer than 336 hours. At 40° C., the presently obtained data are best represented by two curves. At low reaction progress the slope is 1/N=1 (i.e., the reaction grade is N=1). At higher reaction progress the slope changes to ½ (FIGS. 12 and 13)—i.e., the reaction grade is N=2. At 50° C. a second change in slope—i.e., a third reaction grade N>2 is obtained. At 60° C. and 75° C., the slope of 1 was no longer seen.

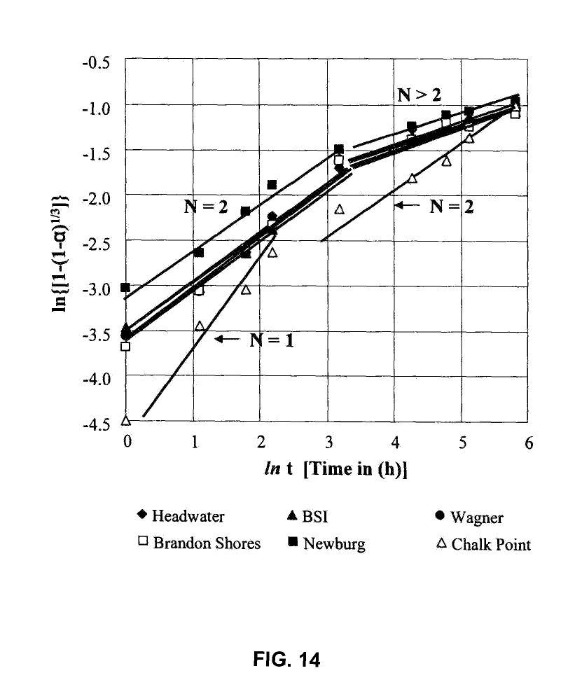

There appear to be three different slopes and the pictures are very similar for all six fly ashes. In FIG. 14 reactivities are plotted for all six fly ashes as they were measured in 7.5 M KOH solutions at 75° C. for up to 336 hours. It was expected that only reaction grades of N=2 and greater would be observed, and this was the case for five out of six fly ashes. Four out of five fly ashes were practically indistinguishable in terms of reactivity. One fly ash (NEW) showed a slightly higher reactivity but the same reaction grades and slopes as the other four fly ashes. CHP fly ash showed a significantly lower reactivity. In this case the reaction between the glass phase and the KOH solution was characterized by two consecutive reaction grades with N=1 and N=2, respectively. The same endpoint, practically complete consumption of the glass, was reached as with the other fly ashes (FIG. 14). The mechanism with N>2 was absent.

Based on the results obtained with the help of Eq. (8) it appears that several processes govern the leaching of fly ash in KOH solution. For the earliest stage of leaching, it is assumed that ‘glass network dissolution’ controls the rate of leaching. Dissolution proceeds linearly with time (N=1). It was presumed that the dissolution mechanism is similar to that proposed for borosilicate nuclear waste glasses in less alkaline solutions (i.e., in the present case stepwise breaking of Si—O—Si and/or Si—O—Al bonds in the aluminosilicate glass network and release of Si(OH)₄ and Al(OH)₄⁻ species into solution). The rate-limiting step could be the detachment of an —O—Si[OH]₃ group [56]. Several authors have shown that [SiO(OH)₃] is the dominant species in solution at around pH=11 and the concentration of [SiO₂(OH)₂]²⁻ increases with increasing pH [57, 58]. Silicon and aluminum hydroxy complexes in highly alkaline solutions are the starting species for geopolymerization [22, 23, 59, 60].

The fact that the glass particles in fly ash come in a distribution of sizes could have affected the results on reaction kinetics shown in FIGS. 12 to 14. The potential implications are discussed below. If all glass particles were of the same size, one would see the shortest possible ranges of transitions (Δα) between two consecutive processes (slopes of the curves in FIGS. 12 to 14). One would expect a mixture of glass particles of different sizes to widen these ranges. FIG. 8 shows a fly ash particle on the lower left side next to the big particle in the middle, and a medium size particle in the upper right edge. The smallest particle shows a tiny residual glass core. The larger one shows a bigger core. The particle in the middle has the biggest unreacted glass core. If different dissolution processes were in progress in each of these particles, the various processes would overlap causing smooth transitions between the curves. The present measurement results show that three consecutive processes are controlling glass dissolution as a function of reaction progress.

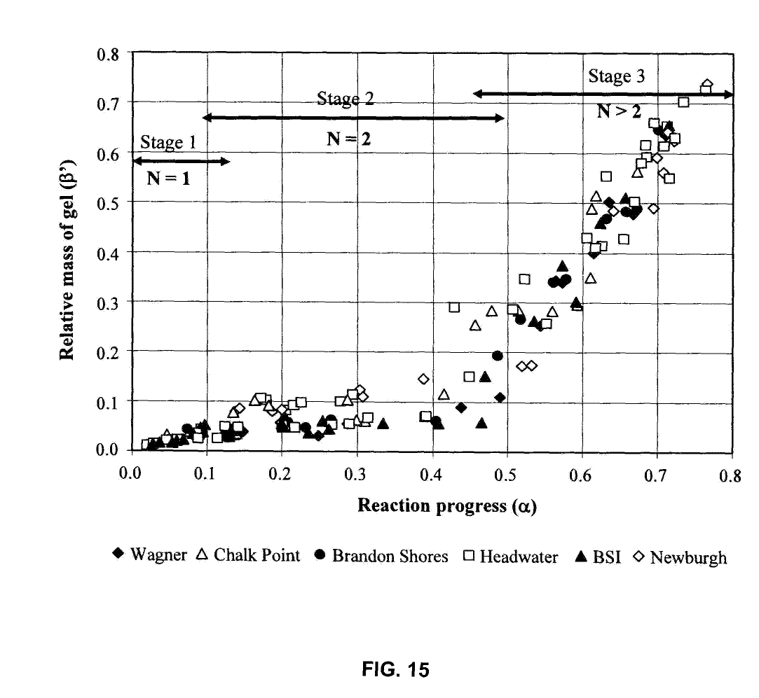

The reaction grades N can be related to what happens at the surface of the glass with increasing reaction progress. FIG. 15 shows the relative mass of gel (β′; Eq. (3)) as a function of reaction progress. Three stages (1 to 3) are identified in FIG. 15 and are related to the reaction grades determined with the help of the processes shown in FIGS. 12, 13, and 14. At the beginning of the leaching process the gel layer is thin and does not constitute a diffusion barrier; glass network dissolution is rate limiting—this is stage 1 in FIG. 15. The reaction grade N is one. Assuming that the mass of the gel is proportional to the thickness of the gel layer, the calculated thickness approaches 0.1 μm at the end of stage 1 (α≈0.1), with an estimated density of the layer of 1.6 g/cm³. Based on DCP-AES measurements the layer was rich in Fe, Ca, Mg and Ti. These glass constituents precipitate as hydroxides (FIG. 10). Most of the reacted Si and Al was dissolved and found in the leachate.

With increasing reaction progress (stage 2) the mass of gel increased, as shown in FIG. 15. Stage 2 is characterized by a reaction grade of N=2, as determined in the processes shown FIGS. 12 and 13. The thin layer of Fe, Ca, Mg, and Ti hydroxides remains in place, while more gel forms on the glass surface, i.e., beneath these hydroxides. FIG. 11 shows that Si and Al became increasingly incorporated into the newly formed gel. From DCP-AES results this gel can be estimated to have a composition of about 30-wt % of SiO₂+Al₂O₃, 30-wt % of Fe₂O₃+TiO₂ and 35-wt % of CaO+MgO. For glass dissolution to continue, OH⁻ and H₂O had to be transported to the glass surface, while dissolved species (e.g., Al(OH)₄⁻ and [SiO₂(OH)₂]²⁻) would either precipitate and form aluminosilicate gel in situ or diffuse into solution or do both. Evidently, transport by diffusion is the step in stage 2 that limits glass dissolution. This was the case until β′ reached a value of about 0.2 g/g-fly ash at a reaction progress of α≈0.45 (FIG. 15).

Further leaching increased the relative mass of gel on the glass surface to 0.75 g/g-fly ash (FIG. 15). The beginning of the relatively steep increase of β′ at α≈0.45 coincided with the change of the slope in FIG. 14 and a corresponding change of the reaction grade from N=2 to N>2. This is the beginning of stage 3 in FIG. 15. Note that zeolite crystallization (Linde F zeolite) began at α≈0.45. Therefore, it appears that the formation of a crust of zeolite on top of the gel layer affected the kinetics of the glass dissolution process. SEM pictures (FIGS. 7 and 8) showed that the zeolite precipitated as a crust, which fully encapsulated the top gel layer rich in Fe, Ca, Mg and Ti, and the aluminosilicate gel layer below. While zeolite crystals precipitate during Stage 3, the aluminosilicate gel layer (the dark gel in FIG. 8) forms in situ and grows inward, until the glass is consumed.

Crystallization of zeolite had a large impact on the mass δ—i.e., the correction to be made to obtain the correct relative mass of fly ash dissolved and reacted in KOH solution (Eq. (2)). The effect of δ on α′ is shown in FIG. 16, in which the relative mass loss of fly ash α′ (Eq. (1)) is plotted as a function of reaction progress. The data comprise almost all results obtained with the six fly ashes at all temperatures. The only selection of results was made with respect to the concentration of the leachant, which was limited to 5 M in this plot. As shown in FIG. 16, all fly ashes yielded very similar results. There was a linear increase in the relative mass of fly ash actually dissolved up to α≈0.45, followed by a linear decrease to almost zero, though α was still increasing. The onset of the decrease coincided with the transition from stage 2 to 3 (i.e., the onset of zeolite precipitation). As mentioned earlier, δ was relatively small at low reaction progress. However, δ increased abruptly at α≈0.45 because substantial amounts of material, not all of it coming directly from the glass, were imported into the surface layer. Without correcting α′ for δ, an apparent decrease of the relative mass of glass dissolved in KOH can be seen. Crystallization of one mol of zeolite Linde F imports one mol of aluminum and silicon each, two moles of oxygen, one mol of potassium, and 1.5 moles of water into the surface layer. As far as glass dissolution is concerned, the crystalline layer was still porous enough for H₂O and OH⁻ to diffuse toward the glass surface (the reaction front); otherwise, glass leaching would have ceased.

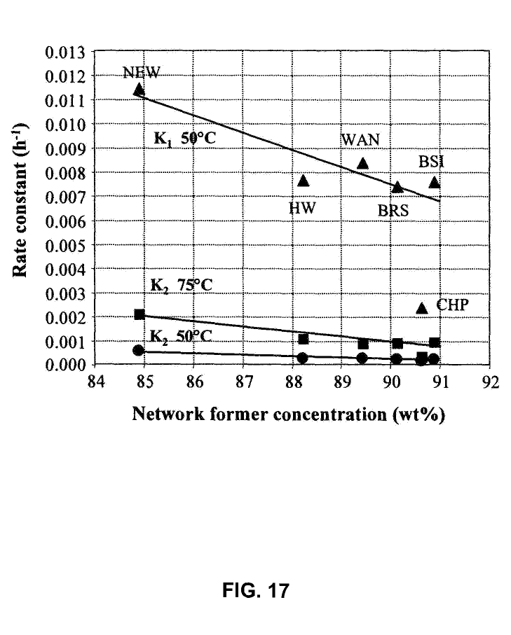

Table 3 shows a compilation of kinetic parameters calculated using Eq. (8) for all fly ash samples leached in 7.5 M KOH solution. During stage 1 (N=1), dissolution of the glass phase was rate controlling in all fly ashes. Fly ashes HW and BSI were investigated more extensively than the other four. K₁ was calculated for three temperatures for HW and BSI. At 50° C. the glass dissolved over 50 times faster than at 20° C. At temperatures above 50° C. the process was too fast to determine K₁ with the presently described experimental technique. Table 3 shows that K₁ values were measured for all fly ashes at 50° C. Glass dissolution was rate controlling for less than 10 hours at this temperature, except for fly ash CHP (72 hours). K₁ characterizes the chemical durability of the glass phase in a fly ash. K₁ depends on glass composition. Since chemical durability and reactivity are inversely proportional, reactivity increases with decreasing concentration of glass network-forming oxides. FIG. 17 shows a trend line (upper curve) suggesting that K₁ is inversely proportional to the content of glass network formers, i.e., chemical durability increases with the sum of SiO₂ and Al₂O₃ (in weight percent) in the glass. The average particle sizes are comparable in all fly ashes, except CHP. Particles of CHP fly ash are on average about twice as large as in the other fly ashes, which explains the outlier.

TABLE 3
Kinetic parameters based on modified Jander equation (leachant 7.5M KOH)

| | | Stage 1 (N = 1) | | Stage 2 (N = 2) | | Stage 3 (N > 2) | |
|---|---|---|---|---|---|---|---|
| | | Observed | | Observed | | Observed | |
| Fly ash | Temp. | between | K₁ | between | K₂ | at | K₃ |
| samples | (° C.) | (hours) | (h⁻¹) | (hours) | (h⁻¹) | (hours) | (h⁻¹) |
| HW1 | 75 | 0-1 | — | 1-72 | 1.3 · 10⁻³ | ≧72 | <1 · 10⁻⁵ |
| HW2 | 75 | — | — | 1-72 | 1.7 · 10⁻³ (10)* | — | — |
| HW3 | | | | | 1.1 · 10⁻³ (5)* | | |
| HW4 | | | | | 6.8 · 10⁻⁴ (3)* | | |
| HW5 | | | | | 5.1 · 10⁻⁴ (1)* | | |
| HW6 | 60 | 0-3 | | 6-72 | 5.3 · 10⁻⁴ | ≧72 | 2.8 · 10⁻⁵ |
| HW7 | 50 | 0-9 | 7.7 · 10⁻³ | 9-120 | 3.0 · 10⁻⁴ | ≧120 | 1.4 · 10⁻⁴ |
| HW8 | 40 | 0-24 | 2.0 · 10⁻³ | 24-168 | 0.8 · 10⁻⁴ | ≧168 | 2.0 · 10⁻⁵ |
| HW9 | 20 | 0-336 | 0.15 · 10⁻³ | — | — | — | — |
| BSI1 | 75 | 0-1 | — | 1-72 | 9.8 · 10⁻⁴ | ≧72 | 0.9 · 10⁻⁵ |
| BSI2 | 60 | 0-3 | — | 6-72 | 4.4 · 10⁻⁴ | ≧72 | 2.8 · 10⁻⁵ |
| BSI3 | 50 | 0-9 | 7.6 · 10⁻³ | 9-120 | 2.8 · 10⁻⁴ | ≧120 | 1.4 · 10⁻⁴ |
| BSI4 | 40 | 0-24 | 2.0 · 10⁻³ | 24-168 | 0.9 · 10⁻⁴ | ≧168 | 1.3 · 10⁻⁵ |
| BSI5 | 20 | 0-336 | 0.15 · 10⁻³ | — | — | — | — |
| NEW1 | 75 | 0-1 | — | 1-24 | 21 · 10⁻⁴ | ≧24 | 1.4 · 10⁻⁵ |
| NEW2 | 50 | 0-9 | 11.5 · 10⁻³ | 9-120 | 5.8 · 10⁻⁴ | ≧120 | 1.3 · 10⁻⁴ |
| BRS1 | 75 | 0-1 | — | 1-72 | 9.5 · 10⁻⁴ | ≧72 | <1 · 10⁻⁵ |
| BRS2 | 50 | 0-9 | 7.4 · 10⁻³ | 9-120 | 2.7 · 10⁻⁴ | ≧120 | 1.6 · 10⁻⁴ |
| WAN1 | 75 | 0-1 | — | 1-72 | 9.3 · 10⁻⁴ | ≧72 | 1.5 · 10⁻⁵ |
| WAN2 | 50 | 0-9 | 8.4 · 10⁻³ | 9-120 | 3.1 · 10⁻⁴ | ≧120 | 1.6 · 10⁻⁴ |
| CHP1 | 75 | 0-9 | 8.2 · 10⁻³ | 9-336 | 3.9 · 10⁻⁴ | ≧336 | — |
| CHP2 | 50 | 0-72 | 2.4 · 10⁻³ | 72-168 | 2.0 · 10⁻⁴ | ≧168 | 1.5 · 10⁻⁴ |

— Not determined.
*KOH molarity

K₁ depends on temperature. Using the Arrhenius equation an activation energy of 102 kJ/mol for K₁ was calculated, i.e., glass network dissolution (HW and BSI fly ash), which is slightly higher than the values reported by Strachan [61] for nuclear waste glass (70-90 kJ/mol) but is in good agreement with the value reported by Barkatt et al. [62] for the SRL glass at high flow rates (100 kJ/mol). K₁ might also depend on pH.

Table 3 shows the K₂ values for stage 2 (N=2)—i.e., for dissolution of the glass phase with diffusion as the rate-limiting step. This process was measurable at all temperatures, except 20° C. because the duration of the experiments herein was limited to two weeks. Again, HW and BSI fly ash were studied in more detail than the other fly ashes. Table 3 shows that K₂ increased by a factor of about 15 between 40° C. and 75° C. for HW and by a factor of 10 for BSI. The diffusion process (K₂) has a lower activation energy than glass network dissolution. The values are Eₐ=66 kJ/mol for HW fly ash and Eₐ=60 kJ/mol for BSI fly ash. These values fall in the range of 41.8 and 83.7 kJ/mol for surface-controlled reaction processes [63] and are in fair agreement with a value of 75 kJ/mol, reported by Rahier et al. [64] for geopolymerization in the metakaolin-potassium silicate solution system. For HW the dependence of K₂ on the OH⁻ molarity at 75° C. was measured (Table 3). More OH′ is transported through the gel layer to the glass surface if more OH⁻ is offered on the surface of the gel layer facing the solution (higher overall KOH molarity). The dependence of K₂ on the OH⁻ molarity suggests that OH⁻ is the diffusing species that limits the rate at which glass can dissolve. An increase in the molarity of KOH by a factor of 10 increased K₂ by a factor of 3 (Table 3).

FIG. 17 shows that K₂ (the two lower curves) also depends on glass composition too, but to a lesser extent than K₁. At 50° C., K₂ decreased by about a factor of two from lowest to highest network former concentration (factor of 5 for K₁). There is no comparison for 75° C. because K₁ at that temperature cannot be measured with the experimental procedure. The dependence of the diffusion process on glass composition could be explained by assuming that the density of the gel increases with increasing concentration of the gel-forming constituents SiO₂ and Al₂O₃, which could slow the transport of OH⁻. Again, CHP fly ash is an outlier.

It is speculated that in stage 3 (N>2), the crust of zeolite precipitated on top of the gel layer lowers the transport rate of OH⁻ more than a gel layer would, except in fly ash CHP, which does not have a zeolite crust. Measurements showed that there is still an effect of the KOH molarity on reactivity of five fly ashes, but less than in stage 2. There is also a dependence of the rate constant on glass composition. K₃ is generally lower at 75° C. than at 50° C.

The glass phases of six fly ashes reacted almost completely within two weeks at the W/S=40, except at the lowest temperature of 20° C. The general reaction pattern was glass network dissolution accompanied increasingly by gel formation and precipitation of a zeolite. The zeolite precipitated as soon as the concentrations of Si and Al reached 0.1 M and 0.05 M, respectively. This was the case at α≈0.45. The Si/Al molar ratio in the zeolite is 1. Condensation of Si and Al species precedes zeolite formation, i.e., formation of enough Si—Al oligomers with a ratio of Si/Al≈1 is needed before the zeolite forms. The literature shows that condensation processes in alkaline solutions depend on the Si/Al molar ratio [59, 60]. Murayama et al. [65] reported that Si and Al species formed aluminosilicate gel followed by zeolite crystallization at high W/S, No zeolite was observed to form from the gel. The boundary between gel and zeolite was sharp. In a few leaching experiments, aluminate was added to the leachant [66]. An aluminate was found to enhance the formation of gel and shifted the appearance of zeolite to lower α values. However, no zeolite crystals were seen in the gel layer.

A detailed analysis of reactivity and the associated processes was possible because there was enough liquid to analyze at the end of the leach tests. At low W/S ratios, e.g., 0.35, typical of geopolymer recipes, gel formation is the dominant process and, according to the results, diffusion (stage 2) is the most likely process controlling reactivity. If fly ash is used to make a geopolymer, the small amount of solution is saturated quickly with Si and Al species leached from the glass phase. Reaction progress is limited because only a limited amount of the glass can react, due to shortage of water. The competition for free water among the glass phase, reacting species, and hydrated cations limits the availability of water for zeolite, whose formation is not possible without incorporating water into its crystals. Studying the leaching process in the absence of a significant amount of leachate is challenging. In this case, characterization of the solid state is more convenient (SEM/EDS, XRD, etc.). At W/S≈0.35, mixing fly ash with, e.g., 7.5 M KOH yields a paste, which hardens within hours at higher curing temperatures (e.g., 75° C.). Respective mixtures at W/S=40 remain thin slurry, which does not harden at all and can be sampled for analysis at any time.

Decreasing the W/S ratio does not change the chemistry of the system dramatically, if the other variables are kept constant. In some embodiments, glass dissolution is relatively small at W/S≈0.35. Chemical attack of the glass network is better described by alteration than dissolution, i.e. direct conversion of glass into gel. For example, at W/S≈0.35, the amount of gel formed at a reaction progress of α=0.3 (75° C., 7.5 M KOH) is the same as that at W/S=40 at α=0.6 with β′≈0.4 g/g-fly ash. To measure α, the leaching reaction was terminated in the reacting paste by washing it with water and then absolute alcohol. Then the gel was dissolved in HCl and the residual mass of glass measured. The onset of massive gel production at W/S≈0.35 starts at α<0.1, whereas for W/S=40, not before α≧0.45.

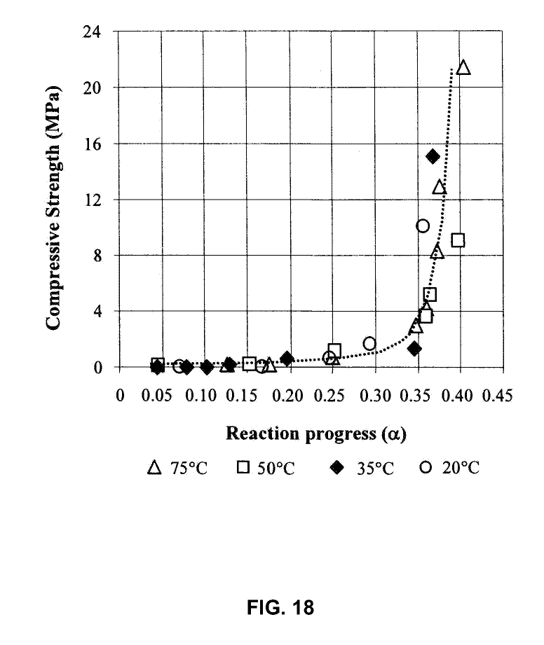

At W/S≈0.35, an interesting relationship was observed between the process underlying N>2 and the compressive strength of the hardening product. This is shown in FIG. 18, where the compressive strength is plotted as a function of reaction progress. Curing times were up to four weeks at 50° C. and 75° C., and up to 24 weeks at the lower temperatures. The plot shows data for fly ash geopolymers prepared with 10 M KOH at 35, 50, and 75° C., respectively. At α<0.3, the geopolymer solid has practically no mechanical strength. At α≈0.35, the reaction grade changes from N=2 to N>2. FIG. 18 shows that α increases only very little beyond 0.35. However, a small increase in a causes a large increase in mechanical durability (compressive strength). In line with the illustration in FIG. 18, the use of reactivity can be used to determine various material properties of materials comprising the fly ash. The property can refer to any property, depending on the context. For example, it can refer to mechanical property, such as compressive property, such as compressive strength. In one embodiment, the reactivity of the fly ash as determined by the method described herein can be further used to determine a mechanical property of at least one of (i) a geopolymer cement, (ii) cement (e.g., Portland cement), and (iii) concrete matrix formed using the fly ash. The material need not be limited to (i)-(iii). For example, the material can be, for example, any product made of or comprised of the fly ash.

Six fly ashes were characterized in terms of composition, glass content, LOI and crystallinity. Reactivities of these six fly ashes were studied in alkaline solution as a function of time, KOH molarity, and temperature at a W/S ratio of 40:1. It can be concluded that:
1. The reactivity of fly ash can be expressed in terms of (i.e., as the sum of) the relative masses of glass phase dissolved, converted into gel, and material precipitated as crystals, as long as only glass constituents are counted. For example, only Si (as SiO₂) and Al (as Al₂O₃) count in Linde F zeolite.
2. Solution analyses and mass loss measurements yield consistent reactivity results.
3. Studying fly ash reactivity in concentrated KOH solutions at a W/S ratio about 100 times higher than those used to make geopolymers allowed measurement of the sequence of processes leading to complete dissolution/alteration of the glass phase.
4. The processes controlling fly ash reactivity can be identified with the help of a modified Jander equation. Depending on the application, the modified equation take the form of Eqs. (6), (7), (8), or a combination thereof
5. Reaction constants can be calculated for the rate-limiting processes. The constants can be analyzed in terms of their dependence on glass composition, temperature and OH-molarity. These dependencies can be used to distinguish fly ashes by their chemical durability (rate of network dissolution), and to identify rate-limiting species in cases of diffusion-controlled reactions.
6. The processes identified and quantified at a high W/S can be used as guidance when analyzing processes at low W/S ratios, which is experimentally more challenging because of the small amount of liquid available for analysis.
7. Reactivity can be related directly to properties of geopolymers such as compressive strength.

### REFERENCES

[1] W. L. Gong, W. Lutze, I. L. Pegg, Low-temperature solidification of radioactive and hazardous wastes (U.S. patent application Ser. No. 11/364,643, filed Feb. 28, 2006).
[2] R. L. Russell, M. J. Schweiger, J. H. Westsik, P. R. Hrma, D. E. Smith, A. B. Gallegos, M. R. Telander, S. G. Pitman, Low temperature waste immobilization testing Vol. 1, PNNL-16052 (Rev. 1), Pacific Northwest National Laboratory, Richland, Wash., September 2006.
[3] F. Pacheco-Torgal, J. Castro-Gomes, S. Jalali, Constr. Build. Mater. 22 (2008) 1305-1314.
[4] J. Davidovits, J. Therm. Anal. 35 (1989) 429-441.
[5] J. Davidovits, J. Therm. Anal. 37 (1991) 1633-1656.
[6] J. Davidovits, J. Mater. Edu. 16 (1994) 91-137.
[7] R. E. Lyon, P. N. Balaguru, A. Foden, U. Sorathia, J. Davidovits, M. Davidovits, Fire Mater. 21 (1997) 67-73.
[8] J. G. S. van Jaarsvelt, J. S. J. Deventer, L. Lorenzen, Miner. Eng. 10 (1997) 659-669.
[9] A. Palomo, M. Grutzeck, M. Blanco, Cem. Concr. Res. 29 (1999) 1323-1329.
[10] D. M. Roy, Cem. Concr. Res. 29 (1999) 249-254.
[11] V. F. F. Barbosa, K. J. D. MacKenzie, C. Thaumaturgo, Int. J. Inorg. Mater. 2 (2000) 309-317.
[12] J. W. Phair, J. S. J. van Deventer, Miner. Eng. 14 (2001) 289-304.
[13] K. Komnitsas, D. Zaharaki, Miner. Eng. 20 (2007) 1261-1277.
[14] P. Duxson, A. Fernandez-Jimenez, J. L. Provis, J. Mater. Sci. 42 (2007) 2917-2933.
[15] D. Khale, R. Chaudhary, J. Mater. Sci. 42 (2007) 729-746.
[16] F. Pacheco-Torgal, J. Castro-Gomes, S. Jalali, Constr. Build. Mater. 22 (2008) 13151322.
[17] W. L. Gong, W. Lutze, I. L. Pegg, Tailored Geopolymer Composite Binders for Cement and Concrete Applications, WO 2010/085537.
[18]H. Xu, J. S. J. van Deventer, Int. J. Miner. Process. 59 (2000) 247-266.
[19] J. C. Swanepoel, C. A. Strydom, Appl. Geochem. 17 (2002) 1143-1148.
[20] J. G. S. van Jaarsvelt, J. S. J. Deventer, G. C. Lukey, Chem. Eng. J. 89 (2002) 63-73.
[21] A. Fernandez-Jimenez, A. Palomo, M. Criado, Cem. Concr. Res. 35 (2005) 1204-1209.
[22] J. L. Provis, P. Duxon, J. S. J. van Deventer, G. C. Lukey, Chem. Eng. Res. Des. 83 (2005) 853-860.
[23] J. L. Provis, J. S. J. van Deventer, Chem. Eng. Sci. 62 (2007) 2309-2317.
[24] A. Fernandez-Jimenez, I. Garcia-Lodeiro, A. Palomo, J. Mater. Sci. 42 (2007) 3055-3065.
[25] M. Criado, A. Fernandez-Jimenez, A. G. de la Torre, Micropor. Mesopor. Mat. 106 (2007) 180-191.
[26] J. L. Provis, G. C. Lukey, J. S. J. van Deventer, Chem. Mater. 17 (2005) 3075-3085.
[27] A. Fernandez-Jimenez, A. Palomo, Cem. Conr. Res. 35 (2005) 1984-1992.
[28] A. Fernandez-Jimenez, A. G. de la Torre, A. Palomo, G. Lopez-Olmo, M. Alonso, M. A. G. Armanda, Fuel 85 (2006) 1960-1969.
[29] M. Criado, A. Fernandez-Jimenez, A. G. de la Torre, M. A. G. Aranda, A. Palomo, Cem. Concr. Res. 37 (2007) 671-679.
[30] X. Querol, N. Moreno, J. C. Umana, A. Alastuey, E. Hernandez, A. Lopez-Soler. F. Plana, Int. J. Coal Geol. 50 (2002) 413-423.
[31] J. G. S. van Jaarsvelt, J. S. J. Deventer, G. C. Lukey, Mater. Lett. 57 (2003) 1272-1280.
[32] A. Fernandez-Jimenez, A. Palomo, Fuel 82 (2003) 2259-2265.
[33] A. Fernandez-Jimenez, A. Palomo, I Sobrados, J. Sans, Micropor. Mesopor. Mater. 91 (2006) 111-119.
[34] A. Palomo, M. M. Alonso, A. Fernandez-Jimenez, J. Am. Ceram. Soc. 87 (2004) 1141-1145.
[35] W. Bumrongjaroen, I. Muller, J. Schweitzer, R. A. Livingston, In: Proceedings of the 2007 World of Coal Ash (WOCA), May 7-10, Covington, Ky., USA.
[36] J. Paya, M. Borrachero, V. J. Month, E. Peris-Mora, F. Amahjour, Cem. Concr. Res. 31 (2001) 41-49.
[37] C. J. Shi, R. L. Day, Cem. Concr. Res. 30 (2002) 607-613.
[38] C. J. Shi, R. L. Day, Cem. Concr. Res. 30 (2000) 51-58.
[39] J. J. Biernacki, P. J. Williams, P. E. Stutzman, ACI Mater. J. 98 (2001) 340-349.
[40] S. Antiohos, S. Tsimas, Cem. Concr. Res. 34 (2004) 769-779.
[41]H. S. Pietersen, A. Fraay, J. M. Bijen, Mat. Res. Soc. Proc. Symp. 176 (1990) 139-157.
[42]H. Xu, J. S. J. van Deventer, Miner. Eng. 15 (2002) 1131-1139.
[43] W. K. W. Lee, J. S. J. van Deventer, Colloid Surface A211 (2002) 49-66.
[44] A. Buchwald, Ch. Kaps, M. Hohmann, In: Proceedings of the 11th International Congress on the Chemistry of Cement (ICCC), Durban, South Africa, 2003, pp. 1238-1246.
[45] Ch. Panagiotopoulou, E. Kontori, Th. Perraki, G. Kakali, J. Mater. Sci. 42 (2007) 2967-2973.
[46] A. Mikuni, R. Komatsu, K. Ikeda, J. Mater. Sci. 42 (2007) 2953-2957.
[47] G. J. McCarthy, A. Thedchanamoorthy, Mat. Res. Soc. Symp. Proc. 136 (1989) 67-76.
[48] A. Fernandez-Jimenez, A. G. de la Torre, A. Palomo, G. Lopez-Olmo, M. M., Alonso, M. A. G. Armanda, Fuel 85 (2006) 625-634.
[49] J. D. Sherman, ACS Symp. Ser. 40 (1977) 30-42.
[50] A. Khawam, D. R. Flanagan, J. Phys. Chem. B110 (2006) 17315-17328.
[51] W. Jander, Z. Anorg. Allgem. Chem. 163 (1927) 1-30.
[52] R. Kondo, K. Lee, M. Diamon, J. Ceram. Soc. (Japan) 84 (1976) 573-578.
[53] P. Dabic, R. Krstulovic, D. Rusic, Cem. Concr. Res. 30 (2000) 1017-1021.
[54] R. Krstulovic, P. Dabic, Cem. Concr. Res. 30 (2000) 693-698.
[55] J. Cabrera, M. F. Rojas, Cem. Concr. Res. 31 (2001) 177-182.
[56] B. Grambow, Mat. Res. Soc. Symp. Proc. 44 (1985) 15-27.
[57] T. W. Swaddle, Coordin. Chem. Rev. 219-221 (2001) 665-686.
[58] M. R. North, T. W. Swaddle, Inorg. Chem. 39 (2000) 2661-2665.
[59] L. Weng, K. Sagoe-Crentsil, J. Mater. Sci. 42 (2007) 2997-3006.
[60] K. Sagoe-Crentsil, L. Weng, J. Mater. Sci. 42 (2007) 3007-3014.
[61] D. M. Strachan, J. Nucl. Mater. 298 (2001) 69-77.
[62] A. Barkatt, B. C. Gibson, P. B. Macedo, C. J. Montrose, W. Sousanpour, M. A., Boroomand, V. Rogers, M. Penafiel, Nucl. Technol. 73 (1986) 140-164.
[63] A. C. Lasaga, Rate laws in chemical reactions, In: A. C. Lasaga, R. J. Kirkpatrick (eds.), Kinetics of Geochemical Processes, Mineral Soc. Am., 1981, pp. 135-169.
[64]H. Rahier, J. Wastiels, M. Biesemans, R. Willlem, G. van Assche, B. van Mele, J. Mater. Sci. 42 (2007) 2982-2996.
[65] N. Murayama, H. Yamamoto, J. Shibata, Int. J. Miner. Process. 64 (2002) 1-17.
[66] C. Chen, W. L. Gong, W. Lutze, I. L. Pegg, J. P. Zhai (to be published).
[67] C. Chen, W. L. Gong, W. Lutze, I. L. Pegg, J. P. Zhai (to be published).

What is claimed:

1. A method of charactering a fly ash composition, comprising determining a reactivity of the fly ash composition in a solution.

2. The method of claim 1, wherein the determination is carried out via at least one of (i) solution analysis and (ii) mass loss measurement.

3. The method of claim 1, wherein the reactivity of the fly ash composition can be expressed in terms of (i) a relative mass of a glass phase of the fly ash dissolved, (ii) a relative mass of a glass phase of the fly ash converted into a gel, (iii) a relative mass of a material precipitated from the fly ash as at least one crystal, or combinations thereof.

4. The method of claim 3, wherein the expression of the reactivity of the fly ash composition is a sum of (i), (ii), and (iii).

5. The method of claim 1, wherein the solution is an alkaline solution.

6. The method of claim 1, wherein the solution comprises at least one alkaline ion.

7. The method of claim 1, wherein a molarity of the alkaline solution is between about 1 to about 10 M.

8. The method of claim 1, wherein the determination is carried out at a temperature of between room temperature and about 90° C.

9. The method of claim 1, wherein a ratio of a liquid to the fly ash composition in the solution is greater than about 1 but less than about 1000.

10. The method of claim 1, wherein a ratio of a liquid to the fly ash composition in the solution is at least 40.

11. The method of claim 1, wherein the fly ash composition comprises a class F fly ash.

12. The method of claim 1, further comprising determining a reaction constant of the fly ash composition.

13. The method of claim 12, wherein the reaction constant is determined via a modified Jander Equation.

14. The method of claim 1, further comprising using the determined reactivity to determine a mechanical property of at least one of (i) a geopolymer cement, (ii) Portland cement, and (iii) concrete matrix formed using the fly ash.

15. A composition, comprising:
(i) a core comprising a fly ash composition,
(ii) a gel layer over at least a portion of the core, wherein the layer comprises at least one oxide, and
(iii) a crust layer over at least a portion of the core, wherein the crust layer comprises at least one crystal.

16. The composition of claim 15, wherein the fly ash composition in the core comprises less than or equal to 10% of a glass phase.

17. The composition of claim 15, wherein the oxide in the gel layer comprises a metal oxide, a silica, or combinations thereof.

18. The composition of claim 15, wherein the oxide in the gel layer comprises silica, alumina, or a combination thereof.

19. The composition of claim 15, wherein the crystal comprises zeolite.

20. The composition of claim 15, wherein the core and the gel layer are substantially free of a crystal.
